(19) United States
(12) Patent Application Publication
(10) Pub. No.: US 2006/0069165 A1
(43) Pub. Date: Mar. 30, 2006

(54) Treating morning hypertension by lowering morning blood pressure levels with propranolol

(76) Inventors: Keith S. Rotenberg, Denville, NJ (US); George Bobotas, Tarpon Springs, FL (US)

Correspondence Address: ARENT FOX PLLC, 1050 CONNECTICUT AVENUE, N.W., SUITE 400, WASHINGTON, DC, 20036, US

(21) Appl. No.: 11/048,052

(22) Filed: Feb. 2, 2005

Related U.S. Application Data
(60) Provisional application No. 60/614,424, filed on Sep. 30, 2004.

(51) Int. Cl. A61K 31/137

(52) U.S. Cl. 514/651

(57) ABSTRACT

This invention relates to a method and kit having a composition for reducing morning blood pressure levels (for treating morning hypertension). Pursuant to this method, a therapeutic amount of β-adrenergic-blocking agent is administered nightly to a person that suffers from blood pressure level attacks such that the blocking agent is released during morning hours when the person is most susceptible to a raised blood pressure level. The kit includes a composition having the therapeutic amount of β-adrenergic-blocking agent that is control released and instructions for administering the composition in evening hours.

This application claims the benefit of U.S. Provisional Patent Application Ser. No. 60/614,424 of Keith S. Rotenberg and George Bobotas, titled “Lowering Morning Blood Pressure Levels with Propranolol” filed Sep. 30, 2004. The entirety of the provisional patent application is incorporated herein by reference.

### FIELD OF INVENTION

The present invention relates to a novel method for treating hypertension by lowering of morning blood pressure levels. Propranolol or a salt thereof is administered to a person to maintain a morning blood pressure level-reducing amount in the person during the morning hours.

### BACKGROUND OF THE INVENTION

Blood pressure has a very definite and reproducible circadian pattern. Blood pressures are highest during the day, lowest during sleep, and rapidly increase from the low nighttime values to the higher daytime values during the period between 4:00 AM and 12:00 noon. It is during the period of rapid early morning blood pressure surge that the peak incidence of non-embolic stroke and myocardial infarction occurs. Although several potential triggers for cardiovascular events during the early morning period have been identified, there is a growing body of evidence suggesting an important association between the morning surge in blood pressure and myocardial ischemia. Studies in which simultaneous Holter monitoring and ambulatory blood pressure monitoring (ABPM) have been performed have demonstrated that surges in blood pressure are frequently followed (within 4-5 minutes) by significant episodes of silent myocardial ischemia. In addition, studies in which adequate blood pressure control is obtained during the early morning period have demonstrated a blunting in the early morning incidence of myocardial infarction. This data would suggest that effective blood pressure control during the early morning period is highly desirable.

This presents a clinical dilemma in that most antihypertensive agents are taken in the morning and are at trough levels during the early morning blood pressure surge, providing the least effective blood pressure control at the time that it is most desirable. Antihypertensives taken at night will usually peak prior to the early morning blood pressure surge coinciding with the time that blood pressure is physiologically at its lowest level and may potentially drive blood pressure too low. Drugs that peak in the early morning, during the period of blood pressure increase, would appear to be optimal. It is for this reason that the inventors set out to develop a chronotherapeutic agent which when dosed at bedtime (e.g., about 10:00 PM) peaks during the period between 6:00 AM and 12:00 noon.

### SUMMARY OF THE INVENTION

The present invention provides a method for utilizing β-adrenergic-blocking agents for the treatment of morning hypertension by control (i.e., lowering) of blood pressure level during morning hours. Thus, it is an object of the present invention to devise a method whereby the need for administration of the blocking agents can be targeted so that the agents are effective to reduce a person's blood pressure level during a particular period in the day, namely the morning hours. Thus, morning hypertension, that is hypertension occurring during the hours between 12:00 midnight and 12:00 noon, preferably between 4:00 AM and 12:00 noon, and more preferably between 6 AM and 11 AM, can be treated.

It has been discovered that the foregoing objectives may be achieved in the treatment of morning hypertension by reducing morning blood pressure levels through the administration of β-adrenergic-blocking agents. In some embodiments, the present invention is directed to a method for lowering morning blood pressure levels in a human comprising administering a β-adrenergic-blocking agent to said human to provide a therapeutically effective amount of said blocking agent in the morning hours of a day. It is preferred in some embodiments that the blocking agent comprises propranolol or a salt thereof.

It has been discovered that the β-adrenergic-blocking agent is desirably provided using a delayed and/or extended release composition suitable for oral administration. This ensures that the β-adrenergic-blocking agent can be taken before a person goes to bed, and the active composition is not released to govern blood pressure level attack until a number of hours later when the person is more likely to be subjected to a raised morning blood pressure level.

In some embodiments, the administration commences in the evening hours of a first day and release of the blocking agent is delayed until a period of time during the following morning hours of a second day. In other variations, a blocking agent is provided to a person in a therapeutically effective amount during the morning hours, for example between 12:00 midnight and 12:00 noon, preferably 4:00 AM to 12:00 noon, or any chosen periods of time therebetween, preferably from 6:00 AM to 11:00 AM.

In accordance with the method of the present invention, these blocking agents successfully reduce morning blood pressure levels.

Accordingly, in some embodiments, the present invention is directed to a kit comprising a composition having a therapeutically effective amount of a β-adrenergic-blocking agent having a delayed release wherein the β-adrenergic-blocking agent (preferably propranolol) is provided in a therapeutically effective amount at least two hours, preferably at least six hours, and more preferably at least eight hours, after administration and instructions indicating that the composition is administered to a human in an evening hour.

Some embodiments of the present invention include a chronotherapeutic composition of a β-adrenergic-blocking agent, wherein the blocking agent has a delayed or controlled release. In variations of this embodiment, the blocking agent is biologically available from two to fourteen hours after administration. Compositions may include a coating or a matrix that facilitates controlled release of the blocking agent in some embodiments.

Any of the embodiments illustrated above and below stand independently or features may be combined to achieve preferred embodiments.

### BRIEF DESCRIPTION OF THE DRAWINGS

FIG. 1 graphically illustrates the steady-state plasma concentrations of InnoPran XL™, in accordance with some embodiments of the present invention.

Other features of the present invention will become apparent from the following detailed description considered in connection with the accompanying drawings, which disclose multiple embodiments of the present invention. It should be understood, however, that the figure is designed for the purpose of illustration only and not as a definition of the limits of the invention. Additional advantages and novel features of the invention will also become apparent to those skilled in the art upon examination of the following or upon learning by practice of the invention.

### DETAILED DESCRIPTION OF THE INVENTION

According to the present invention, it has been discovered that a β-adrenergic-blocking agent is effective for treating morning blood pressure levels such that the levels are reduced in comparison to what the levels would be in the absence of the blocking agent. The β-adrenergic-blocking agent can be administered prior to bedtime (e.g. between 8:00 PM and 12:00 midnight) to be effective in the early hours of each morning so that the biological availability of the effects of the blocking agent will last during the morning time period during which a person is prone to raised blood pressure levels. Preferably, the β-adrenergic-blocking agent is in a delayed and/or controlled release form. Thus, the blocking agent can be taken by a person before going to bed, and the blocking agent will not become biologically available until it is released over a period of the morning hours, such as for example from 12:00 midnight to 12:00 noon, preferably 4:00 AM to 12:00 noon, or any chosen periods of time there between, preferably from 6:00 AM to 11:00 AM.

The present invention involves a targeted administration during a specific period in the day, in particular during the morning hours. The drug is administered such that it is effective primarily during these hours. This increases the availability of this form of therapy for many classes of individuals suffering from raised morning blood pressure levels. In addition, it has been observed that the incidents of fatigue is reduced according to the administration regime of the invention, as compared to other products and administration regimes, for example Inderal® LA.

For example, for the targeted method of the present invention, the preferred dose is normally from about 60 to about 160 mg, preferably about 80 to about 120 mg, and most preferably about 80 or about 120 mg, of propranolol hydrochloride per day.

The preferred administration of the β-adrenergic-blocking agent is once per day, in the evening (e.g., between 6 PM and 12 midnight), preferably before bedtime (e.g., between 8 PM and 12 midnight, preferably about 10 PM).

It is important to ensure the entry of the β-adrenergic-blocking agent into the bloodstream at the appropriate time of day, namely during morning hours.

Minor amounts of other ingredients such as tonicity agents (e.g. NaCl), pH adjusters (e.g., a base such as NaOH, acids such as citric), emulsifiers or dispersing agents, buffering agents, preservatives, wetting agents, thickening agents (e.g. polyvinyl alcohol) and gelling agents (e.g. polaxamer) may also be present. Particularly preferred compositions contain sufficient amounts of the foregoing and/or other ingredients to be a substantially isotonic and/or buffered to a physiologically acceptable pH.

As previously discussed, the efficacy of a β-adrenergic-blocking agent is dependent upon its presence at the desired site of drug activity. This is commonly reflected by its concentration in the blood of the subject being treated. It is therefore particularly significant that the β-adrenergic-blocking agent is characterized by a pronounced blood concentration only during certain hours, preferably morning hours.

To maximize its efficacy, it is desirable that the presence of β-adrenergic-blocking agent in a therapeutically effective amount be maintained over a substantial period of time. Thus, it is preferred to sustain an appropriate blood concentration of blocking agent for at least two morning hours, and preferably three, four, five, six or seven or more morning hours. This may be ensured by using a delayed and/or controlled release formulation.

Any effective controlled and/or delayed release enhancing compounds can be utilized in the formulation. Also, the delayed release mechanism and/or components are preferably in the form of a coating, but can take the form of any other effective vehicle, while the controlled release mechanism and/or components are preferably in the form or a coating or a matrix, but can also be in the form of an any other effective vehicle.

The controlled and delayed release formulations can be made of two or more components. A first part is a central core, which can contain the blocking agent or can be coated with a coating that contains the blocking agent, for example in association with conventional excipients. Another coating, such as a polymeric coating, envelops or substantially envelops the central core. This coating is responsible for giving the blocking agent its particular controlled and/or delayed release characteristics.

The central core may be prepared by a number of techniques known in the art. Typically, the blocking agent is bound to an inert carrier with a conventional binding agent. The inert carrier is typically a starch or sugar sphere. Sugar spheres are preferred, but any pharmaceutically acceptable inert carrier may be utilized.

The binding agent that is used to secure the blocking agent can be any of the known binding agents. Examples of suitable lubricants that can be used include white wax, castor oil, palmitic acid, stearic acid, mineral oil, polyethylene glycol, etc. Examples of suitable coating agents that can be used include ethyl cellulose, methylcellulose, carboxymethylcellulose, hydroxypropymethylcellulose, polyvinylpyrrolidone, polymerized acrylates, etc. Other conventional pharmaceutical excipients may be incorporated into the binding agent.

The polymeric coating is responsible for giving the blocking agent its particular release characteristics. The coating may be produced, for example, from polymerized acrylates or copolymers of acrylic acid and methacrylic acid or esters of either monomer (hereinafter polymerized acrylates). The polymeric coating of the delayed release pellet may also be prepared from one of the organosiloxane oral coating materials known in the art such as polydimethylsiloxane, polydiethylsiloxane, etc.

Polymerized acrylates as well as copolymers of acrylic acid and methacrylic acid or esters of either monomer are known in the art and are available from many commercial sources. Examples of such copolymers include poly(methyl methacrylate), poly(ethyl methacrylate), poly(butyl methacrylate), poly(isobutyl methacrylate), poly(isobutyl methacrylate), poly(phenyl methacrylate) etc. The polymeric coating may optionally contain a sufficient quantity of a suitable plasticizer. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate.

The polymeric coating may also be made from a variety of coating materials that are typically utilized in the pharmaceutical arts. The coating may be manufactured from a variety of water insoluble polymers such as, for example, ethylcellulose, cellulose acetate, cellulose propionate, cellulose acetate butyrate, polyethylene, polypropylene, polyethylene oxide, polyvinyl acetate, polyvinyl chloride, etc. A minor proportion of a water-soluble polymer may also be included in the polymeric coating. Examples of such polymers include methylcellulose, hydroxypropyl cellulose, polyethylene glycol, polyvinyl pyrrolidone, etc. These coatings may also include conventional excipients such as plasticizers, antifoaming agents, antiadherants, etc.

The polymeric coating may be applied to the central core using methods and techniques known in the art. Typically a suspension, emulsion, or solution of the polymeric coating is prepared as is known in the art. The amount of fluidized polymeric coating required in the coating process may be readily calculated depending upon the amount of polymeric coating desired. The fluid polymeric coating may be applied to the central core by a number of coating techniques known in the art. Examples of suitable coating devices include fluid bed coaters, pan coaters, etc.

The sustained-release forms of administration according to the invention can also contain the blocking agent in a sustained-release matrix, preferably as a uniform distribution.

Matrix materials that can be used are physiologically compatible, hydrophilic materials known to those skilled in the art. The hydrophilic matrix materials used are preferably polymers and particularly preferably cellulose ethers, cellulose esters and/or acrylic resins. The matrix materials used are very particularly preferably ethyl cellulose, hydroxypropyl methyl cellulose, hydroxypropyl cellulose, hydroxymethyl cellulose, poly(meth)acrylic acid and/or derivatives thereof such as their salts, amides or esters.

Other preferred matrix materials are those consisting of hydrophobic materials such as hydrophobic polymers, waxes, fats, long-chain fatty acids, fatty alcohols or corresponding esters or ethers, or mixtures thereof. The hydrophobic materials used are particularly preferably C₁₂-C₃₀ fatty acid mono- or diglycerides and/or C₁₂-C₃₀ fatty alcohols and/or waxes, or mixtures thereof.

It is also possible to use mixtures of the above-mentioned hydrophilic and hydrophobic materials as the sustained-release matrix material.

The sustained-release matrix can be prepared by the conventional methods known to those skilled in the art.

Those skilled in the art will be aware that a therapeutically effective amount of a particular β-adrenergic-blocking agent will vary with the particular drug as well as the type, age, size, weight and general physical condition of the subject. The amount will also vary dependent upon the particular therapeutic effect desired.

Any of the β-adrenergic-blocking agents known in the art may be utilized in accordance with the present invention. This includes blocking agents in their basic states or as their acid addition salts. Certain β-adrenergic-blocking agents are, however, preferred. These include propranolol, nadolol, timolol, metoprolol, atenolol, labetolol, pindolol, oxprenolol and their salts. Of these, propranolol (or a salt thereof), particularly propranolol hydrochloride, is most preferred.

The most preferred embodiments of this invention utilize, as the blocking agent, the product InnoPran XL™ produced by Reliant Pharmaceuticals LLC. InnoPran XL™ is in the form of capsules that contain 80 or 120 mg of active ingredient propranolol hydrochloride along with sugar spheres, ethylcellulose, povidone, hypromellose phthalate, diethyl phthalate, hypromellose, polyethylene glycol, gelatin, titanium dioxide and black iron oxide. The 120 mg capsules also contain yellow iron oxide.

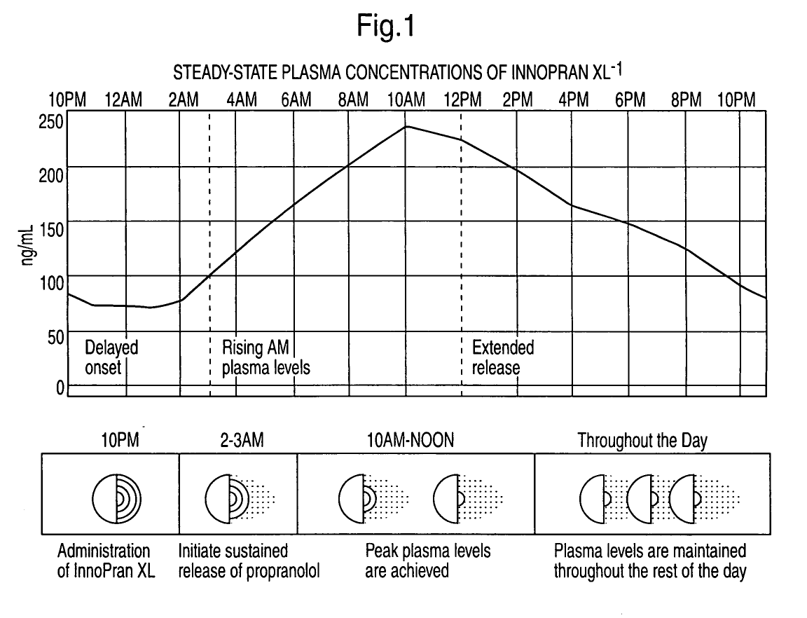

FIG. 1 presents an exemplary graphical illustration of the plasma concentrations of InnoPran XL™. As shown in FIG. 1, InnoPran XL™ is administered at 10 PM. The plasma concentrations remain generally stable for several hours. After approximately five hours, i.e., the delayed onset, the plasma levels increase. At approximately 10 AM, the plasma concentration of InnoPran XL™ peaks. Peak plasma levels are achieved between approximately 10 AM and 12 PM. Over the course of approximately the next twelve hours, the plasma concentration of InnoPran XL™ steadily declines.

A clinical trial report further demonstrating the effectiveness of the present invention is described hereafter. A multi center, prospective, double-blind, double dummy, randomized, blinded endpoint crossover study comparing InnoPran XL™ to Inderal® LA in patients with stage I and stage II hypertension

Objectives: Primary objectives were to characterize the pharmacokinetics of InnoPran XL™ 120 mg and Inderal® LA 120 mg and to compare blood pressure reductions during the hours of 6:00 AM and 12:00 noon, in patients treated either with InnoPran XL™ 120 mg or Inderal® LA 120 mg.

Secondary objectives were to compare the pharmacokinetics and the effects on blood pressure of InnoPran XL™ 120 mg and Inderal® LA 120 mg during predefined time periods and to assess the safety of InnoPran XL™ 120 mg and Inderal® LA 120 mg in patients with Stage I and Stage II Hypertension.

Methodology: This was a multicenter, prospective, double-blind, double dummy, randomized blinded endpoint crossover study that compared InnoPran XL™ to Inderal® LA. After 4 weeks of a single-blind placebo phase, patients were randomized to InnoPran XL™ 80 mg or Inderal® LA 80 mg for 1 week. Following one week of this initial treatment, patient on Inderal® LA 80 mg were force-titrated to Inderal® LA 120 mg for a period of 4 weeks, and patients on InnoPran XL™ 80 mg were force-titrated to InnoPran XL™ 120 mg for a period of 4 weeks.

After completion of this 4-week period, each patient had ambulatory blood pressure monitoring (ABPM) applied for 24 hours. Within 48 hours of removal of the ABPM, patients were admitted to the research center for 34-hour pharmacokinetic (PK) analysis and blood pressure (BP) measurements. At the end of the 34-hour PK period, patients crossed-over to the opposite arm for a period of 4 weeks. Patients on InnoPran XL™ 120 mg received Inderal® LA 120 mg and patients on Inderal® LA 120 mg received InnoPran XL™ 120 mg.

After completion of this 4-week period, each patient had an ABPM applied for 24 hours. Within 48 hours of removal of the ABPM, patients were admitted to the research center for a repeat of the 34-hour PK analysis and BP measurements. Patients were then weaned off their study medication at 80 mg for 3 days and then the patients exited the study.

Duration of treatment: The total duration of the study was approximately 13 weeks. The duration of the double-blind phase was 9 weeks. This was preceded by a 4-week single-blind phase and followed by a 3-day tapering phase.

Criteria for Evaluation:

Efficacy: To characterize the pharmacokinetic profile of InnoPran XL™ 120 mg and Inderal® LA 120 mg and to compare blood pressure reductions during the hours of 6:00 AM and 12:00 noon, in patients treated either with InnoPran XL™ 120 mg or Inderal® LA 120 mg. Other pharmacokinetic and/or ABPM evaluations included the time period that corresponds to the peak incidence of cardiovascular events (6:00 AM to 12:00 noon), trough period, delayed release period (InnoPran XL™) vs. trough period (Inderal® LA), mean 24 hour ABPM, and early morning BP surge.

Results: Sixty-three patients were screened and a total of 44 patients were randomized, comprising the safety evaluable population. Forty-one patients were included in the Intent-to-Treat population, which included 38 per-protocol patients. There were no statistically significant differences (p>0.2349) in age, gender, race, mean seated and mean daytime blood pressure at baseline between the two treatment groups. Almost all patients (43/44 or 97.7%) were on antihypertensive medication prior to study enrollment.

Efficacy: Characterization of the 24-hour pharmacokinetic profile of InnoPran XL™ 120 mg and Inderal® LA 120 mg showed a significant difference involving Tₘₐₓ and Tₘᵢₙ for InnoPran XL™ as compared to Inderal® LA. Between 6:00 AM and 12:00 noon, InnoPran XL™ exhibited a significant larger area under the plasma concentration time curve and higher average plasma concentration than Inderal® LA. Overall change in diastolic and systolic blood pressure adjusted for covariates showed a significant difference in diastolic blood pressure (p=0.0470) and systolic blood pressure (p=0.0337) between the treatment groups representing a greater reduction in diastolic and systolic blood pressure in patients on InnoPran XL™ during these time intervals.

Having thus described presently preferred embodiments of the present invention, it will be appreciated that the objects of the invention have been achieved, and it will be understood by those skilled in the art that changes in construction and widely differing embodiments and applications of the invention will suggest themselves without departing from the spirit and scope of the present invention. The disclosure and description herein are intended to be illustrative and are not in any sense limiting of the invention.

What we claim:

1. A method for lowering morning blood pressure levels in a human, comprising:
administering a β-adrenergic-blocking agent to said human to provide a therapeutically effective amount of said blocking agent in the morning hours of a day.

2. The method of claim 1, wherein the blocking agent comprises propranolol or a salt thereof.

3. The method of claim 1, wherein the administration commences in the evening hours of a first day and release of the blocking agent is delayed until a period of time during the following morning hours of a second day.

4. The method of claim 1, wherein the administration occurs once daily.

5. The method of claim 1, wherein the administration occurs orally.

6. The method of claim 3, wherein the blocking agent is administered each evening for a plurality of evenings.

7. The method of claim 2, wherein the blocking agent comprises a salt of propranolol.

8. The method of claim 1, wherein the blocking agent is administered in a pill or capsule composition.

9. The method of claim 8, wherein the pill or capsule is coated with a delayed-release membrane and/or with a controlled-release membrane.

10. The method of claim 9, wherein the pill or capsule is administered in the evening, and a therapeutically effective amount of the blocking agent is provided during the morning hours of the day.

11. The method of claim 3, wherein the evening hours range from about 8 pm to 12 midnight.

12. The method of claim 1, wherein the blocking agent is released continuously for at least two hours between the hours of 4 AM and 12 noon.

13. The method of claim 1, wherein the blocking agent is released continuously for at least two hours between the hours of 6 AM and 11 AM.

14. The method of claim 2, wherein the blocking agent administered comprises from about 50 to about 450 mg.

15. The method of claim 2, wherein the blocking agent administered comprises from about 60 to about 320 mg.

16. The method of claim 2, wherein the blocking agent administered comprises from about 80 to about 160 mg.

17. The method of claim 2, wherein the blocking agent administered comprises about 80 mg of propranolol or salt thereof.

18. The method of claim 2, wherein the blocking agent administered comprises about 120 mg of propranolol or salt thereof.

19. The method of claim 1, wherein the blocking agent sustains a therapeutically effective blood concentration for at least two morning hours.

20. The method of claim 1, wherein the blocking agent sustains a therapeutically effective blood concentration for at least four morning hours.

21. The method of claim 1, wherein the blocking agent sustains a therapeutically effective blood concentration for at least seven morning hours.

22. The method of claim 1, wherein the blocking agent obtains peak plasma levels ten to sixteen hours after administration.

23. The method of claim 1, wherein the blocking agent obtains peak plasma levels twelve to fourteen hours after administration.

24. The method of claim 1, wherein a mean systolic blood pressure is lowered at least 5 mmHg and a mean diastolic blood pressure is lowered at least 5 mmHg during the morning hours, as compared to a time immediately preceding administration.

25. The method of claim 1, wherein the mean systolic blood pressure is lowered at least 9 mmHg and a mean diastolic blood pressure is lowered at least 9 mmHg during the morning hours, as compared to a time immediately preceding administration.

26. A kit for lowering morning blood pressure, the kit comprising:
a composition comprising a therapeutically effective amount of a β-adrenergic-blocking agent having a delayed release wherein the β-adrenergic-blocking agent is provided in a therapeutically effective amount at least two hours after administration; and
instructions indicating that the composition is administered to a human in an evening hour.

27. The kit according to claim 26, wherein the blocking agent obtains peak plasma levels ten to sixteen hours after an administration to a human.

28. The kit according to claim 27, wherein the blocking agent obtains peak plasma levels twelve to fourteen hours after an administration to a human.

29. The kit according to claim 26, wherein the blocking agent comprises propranolol or a salt thereof.

30. The kit according to claim 26, the composition further compromises one or more components in a form of a coating or a matrix.

31. The kit according to claim 30, wherein the one or more components comprise:
a central core comprising the blocking agent;
a coating agent enveloping the central core; and
optionally an inert carrier bound together with the central core using a binding agent.

32. The kit according to claim 26, wherein the composition is in a pill or a capsule.

33. The kit according to claim 29, wherein the therapeutically effective amount of propranolol or a salt thereof is 80 mg.

34. The composition according to claim 29, wherein the therapeutically effective amount of propranolol or a salt thereof is 120 mg.
